(19) United States
(12) Patent Application Publication
(10) Pub. No.: US 2002/0095127 A1
(43) Pub. Date: Jul. 18, 2002

(54) Controlled delamination of laminate structures having enclosed discrete regions of a material

(75) Inventors: Jeffrey E. Fish, Dacula, GA (US); Jennifer A. Griffiths, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/027,246

(22) Filed: Dec. 20, 2001

Related U.S. Application Data
(60) Provisional application No. 60/259,134, filed on Dec. 28, 2000.

(51) Int. Cl. A61F 13/15

(52) U.S. Cl. 604/368; 604/385.01

(57) ABSTRACT

A laminate structure that is formed from a first substrate, a second substrate, and discrete regions of particles sandwiched therebetween is provided. In particular, the first and second substrates are bonded together at certain portions such that bonded portions and unbonded portions are formed. The unbonded portions form pockets that contain the particles. The pockets have a length-to-width ratio of greater than about 2. The resulting laminate structure of the present invention can have inner regions that delaminate upon the application of a certain force (e.g., swelling of superabsorbent particles), as well as perimeter regions that do not substantially delaminate upon the application of the same force.

### RELATED APPLICATIONS

This application claims priority to U.S. Provisional Application Serial No. 60/259,134, filed on Dec. 28, 2000.

### BACKGROUND OF THE INVENTION

In order to enhance the functionality of a laminate material, it is often desired to enclose certain particles within the laminate. For example, to enhance the absorbency of a disposable diaper, superabsorbent particles can be enclosed within pockets formed by a laminate material of the diaper to inhibit undesirable shifting, channeling, gel blocking, dusting, or settling during use. To accomplish such particle deposition within pockets, a variety of techniques have been developed. For instance, U.S. Pat. Nos. 4,327,728 to Elias and 4,381,783 to Elias describe an absorbent article that includes at least one pocket containing a uniform admixture of discrete superabsorbent particles and discrete particles.

When incorporating superabsorbent particles or other materials that swell or expand when contacted with a liquid, however, some conventional techniques for enclosing the particles within pockets have proven inadequate. For example, in some instances, as the particles absorb water, they swell to such an extent that they begin to abut the lower surface of the substrates. Thereafter, as the particles continue to swell to at or near saturation, they exert increased forces on the surface of the substrate, which eventually causes the substrate to rupture. Besides causing the substrates to rupture, the particles often swell to such an extent that they prevent liquids from flowing to other, unswollen particles within the pockets.

In response to these problems, various techniques have been developed. For instance, U.S. Pat. No. 5,983,650 to Baer, et al. describes an absorbent core that contains layers that are free of wood pulp or other cellulosic materials. The core contains a superabsorbing polymer contained in flat pockets defined by a bonding gridwork. As liquid is applied to an area of the laminate, the polymer particles within the pocket swell. As the particles swell, the bonded areas form three-dimensional channels and allow excess liquid in one location to flow quickly to adjacent and more remote pockets. In some applications, the forces generated by the swollen SAP particles can or will cause disruption of at least a portion of a seal line.

Nevertheless, although the techniques described above have represented some improvements, these techniques are still inefficient in their use of the particles contained within the pockets. As such, a need currently exists for a method of an improved and more efficient method of enclosing particles within pockets of a laminate.

### SUMMARY OF THE INVENTION

In accordance with one embodiment of the present invention, a laminate structure is provided that includes a first substrate and a second substrate. In one embodiment, for example, the substrates can contain thermoplastic polymers that are fused together to form bonded portions and unbonded portions located between the bonded portions.

The unbonded portions of the laminate structure define elongated pockets containing discrete regions of particles. The elongated pockets have a length-to-width ratio of greater than about 2. Moreover, in some embodiments, the elongated pockets have a length-to-width ratio of between about 4 to about 100, and in some embodiments, between about 6 to about 10.

In addition, the bonded portions define at least one perimeter region and at least one inner region. The inner region is bonded to an extent such that it is capable of delaminating upon the application of a force thereto. For example, in some instances, superabsorbent particles can be utilized that swell upon being contacted with water. Such swelling can cause a force to applied against the inner region of the laminate structure, thereby delaminate the structure at that region. Moreover, in one embodiment, the perimeter region is bonded to a greater extent than the inner region such that the perimeter region does not substantially delaminate upon the application of the same force. For example, in some embodiments, the perimeter regions can be bonded to have a strength that approximates the strength of the substrates.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a schematic view of the steps for forming one embodiment of a laminate structure of the present invention in which FIG. 1A illustrates particles deposited onto a first substrate, FIG. 1B illustrates a second substrate placed over the particles, and FIG. 1C illustrates the two substrates bonded together;

FIG. 2 is a plan view of one embodiment of a laminate structure formed in accordance with the present invention;

FIG. 3 is a plan view of the laminate structure illustrated in FIG. 2 in which the inner regions of the laminate structure have delaminated; and

FIG. 4 is side view of one embodiment of a laminate structure of the present invention;

FIG. 5 is a side view of the laminate structure illustrated in FIG. 4 in which the inner regions of the laminate structure have delaminated;

FIG. 6 is a schematic illustration of one technique that can be utilized to form one embodiment of a laminate structure of the present invention;

FIG. 7 is a schematic illustration of a bonding plate used to form the laminate structure in the Examples; and

FIGS. 8-13 are stress-strain curves developed for the samples of Example 2, in which the load (pounds) was determined as a function of extension (inches).

Repeat use of reference characters in the present specification and drawings is intended to represent same or analogous features or elements of the invention.

### DETAILED DESCRIPTION OF REPRESENTATIVE EMBODIMENTS

### Definitions

As used herein the phrase “bonded carded web” refers to webs that are made from staple fibers which are sent through a combing or carding unit, which separates or breaks apart and aligns the staple fibers to form a nonwoven web. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well-known bonding method, particularly when using bicomponent staple fibers, is through-air bonding.

As used herein, “meltblown fibers” refers to fibers formed by extruding a molten thermoplastic material through a plurality of fine, usually circular, die capillaries as molten threads or filaments into converging high velocity, usually hot gas (e.g., air) streams which attenuate the filaments of thermoplastic material to reduce their diameter, which may be to microfiber diameter. Thereafter, the meltblown fibers are carried by the high velocity gas stream and are deposited on a collecting surface to form a web of nearly randomly disbursed meltblown fibers. Such a process is disclosed, for example, in U.S. Pat. No. 3,849,241 to Butin et al. For example, meltblown fibers may be microfibers that are continuous or discontinuous and can have a diameter smaller than 10 microns.

As used herein, the term “nonwoven web” or “nonwoven” refers to a web having a structure of individual fibers or threads which are interlaid, but not in an identifiable manner as in a knitted fabric. Nonwoven webs or fabrics have been formed from many processes, such as, for example, meltblowing processes, spunbonding processes, and bonded carded web processes. The basis weight of nonwoven fabrics is usually expressed in ounces of material per square yard (“osy”) or grams per square meter (“gsm”) and the fibers diameters are usually expressed in microns. (Note that to convert from “osy” to “gsm”, multiply “osy” by 33.91).

As used herein, the phrases “pattern unbonded”, “point unbonded”, or “PUB” generally refer to a fabric pattern having continuous thermally-bonded areas defining a plurality of discrete unbonded areas. The fibers or filaments within the discrete unbonded areas are dimensionally stabilized by the continuously bonded areas that encircle or surround each unbonded area. The unbonded areas are specifically designed to afford spaces between fibers or filaments within the unbonded areas. A suitable process for forming the pattern-unbonded nonwoven material of this invention, such as described in U.S. Pat. No. 5,962,117, includes passing a heated nonwoven fabric (e.g., nonwoven web or multiple nonwoven web layers) between calendar rolls, with at least one of the rolls having a bonding pattern on its outermost surface comprising a continuous pattern of land areas defining a plurality of discrete openings, indentions, apertures, or holes. Each of the openings in the roll (or rolls) defined by the continuous land areas forms a discrete unbonded area in at least one surface of the resulting nonwoven fabric in which the fibers or filaments are substantially or completely unbonded. Alternative embodiments of the process include pre-bonding the nonwoven fabric or web before passing the fabric or web within the nip formed by the calender rolls.

As used herein, “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. Nos. 4,340,563 to Appel et al., 3,692,618 to Dorschner et al., 3,802,817 to Matsuki et al., 3,338,992 to Kinney, 3,341,394 to Kinney, 3,502,763 to Hartman, and 3,542,615 to Dobo et al., Spunbond fibers are generally not tacky when they are deposited on a collecting surface. Spunbond fibers are generally continuous and have diameters larger than about 7 microns, and more particularly, between about 10 and 40 microns.

As used herein, the term “superabsorbent material” (SAM) generally refers to any substantially water-swellable, water-insoluble material capable of absorbing, swelling, or gelling, at least about 10 times its weight, and in some embodiments at least about 30 times its weight, in an aqueous solution, such as water. Moreover, a superabsorbent material can generally absorb at least about 20 grams of an aqueous solution per gram of the SAM, particularly at least about 50 grams, more specifically at least about 75 grams, and more particularly between about 100 grams to about 350 grams of aqueous solution per gram of SAM. Some suitable superabsorbent materials that can be used include inorganic and organic materials. For example, some suitable inorganic superabsorbent materials can include absorbent clays and silica gels. Moreover, some suitable superabsorbent organic materials include natural materials, such as agar, pectin, guar gum, etc., as well as synthetic materials, such as synthetic hydrogel polymers. For example, one suitable superabsorbent material is FAVOR 880 available from Stockhausen, Inc., located in Greensboro, N.C.

As used herein, the phrase “thermal point bonding” generally refers to passing a fabric (e.g., fibrous web or multiple fibrous web layers) or webs to be bonded between heated calendar rolls. One roll is usually patterned in some way so that the entire fabric is not bonded across its entire surface, and the other roll is usually smooth. As a result, various patterns for calendar rolls have been developed for functional as well as aesthetic reasons. One example of a pattern that has points is the Hansen-Pennings or “H&P” pattern with about a 30% bond area with about 200 pins/square inch as taught in U.S. Pat. No. 3,855,046. The H&P pattern has square point or pin bonding areas. Another typical point bonding pattern is the expanded Hansen-Pennings or “EHP” bond pattern which produces a 15% bond area. Another typical point bonding pattern designated “714” has square pin bonding areas wherein the resulting pattern has a bonded area of about 15%. Other common patterns include a diamond pattern with repeating and slightly offset diamonds with about a 16% bond area and a wire weave pattern looking as the name suggests, e.g. like a window screen, with about an 18% bond area. Typically, the calender imparts from about 10% to about 30% bonded area of the resulting fabric. As is well known in the art, the point bonding holds the resulting fabric together.

As used herein, “ultrasonic bonding” generally refers a process performed, for example, by passing a substrate between a sonic horn and anvil roll, such as illustrated in U.S. Pat. No. 4,374,888 to Bornslaeger.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents.

In general, the present invention is directed to a laminate structure that contains elongated pockets formed by bonding at least two substrates together. The elongated pockets contain discrete regions of particles (e.g., superabsorbent materials). It has been discovered that a laminate structure formed according to the present invention can provide a more effective utilization of the particles contained therein than various prior art methods. For example, in some embodiments, the pockets can have a certain length-to-width ratio such that the pockets can more easily delaminate in the height direction upon the application of a force. Specifically, it has been discovered that such elongated pockets can allow for forces created by the swelling of a particle to be applied to a greater extent in the width direction of a pocket than in the length direction, thereby making it more likely that the laminate structure will delaminate at the inner bonded regions than the perimeter bonded regions of the laminate structure.

The laminate structure of the present invention can generally be formed from two or more substrates that can each contain one or more layers. For example, the substrates may be hydrophobic or hydrophilic. Moreover, the substrates used in the present invention can also be made from a variety of different materials, so long as at least a portion of two or more of the substrates are bondable when subjected to thermal, ultrasonic, adhesives, or other similar bonding techniques. For instance, in some embodiments, the substrates can be generally free of cellulosic materials to enhance the ability of the substrates to be bonded together. Moreover, it is also typically desired that the substrates possess enough strength that they will not substantially rupture upon the swelling of particles contained therein. For example, a substrate used in the present invention can be formed from films, nonwoven webs, woven fabrics, knitted fabrics, or combinations thereof (e.g., nonwoven fabric laminated to a film).

As stated, in one embodiment, the substrates can be formed from one or more nonwoven webs. In some instances, the basis weight and/or the thickness of the nonwoven webs may be selected within a certain range to enhance the flexibility of the laminate structure. For example, it has been discovered that, in some instances, an increase in the thickness of a particular substrate can cause the stiffness of the substrate to increase to the third power with thickness. Thus, in some embodiments, the thickness of the nonwoven webs can be less than about 0.1 inches, in some embodiments between about 0.005 inches to about 0.06 inches, and in some embodiments, between about 0.015 inches to about 0.03 inches. Moreover, in some embodiments, the basis weight of the nonwoven webs can be less than about 5 ounces per square yard, in some embodiments, between about 0.5 to about 4 ounces per square yard, and in some embodiments, between about 0.5 to about 2 ounces per square yard.

Typically, the nonwoven webs used in the present invention contain synthetic fibers or filaments. The synthetic fibers or filaments may be formed from a variety of thermoplastic polymers. For example, some suitable thermoplastics include, but are not limited, poly(vinyl) chlorides; polyesters; polyamides; polyolefins (e.g., polyethylene, polypropylenes, polybutylenes, etc.); polyurethanes; polystyrenes; poly(vinyl) alcohols; copolymers, terpolymers, and blends of the foregoing; and the like.

Some suitable polyolefins, for example, may include polyethylenes, such as Dow Chemical's PE XU 61800.41 linear low density polyethylene (“LLDPE”) and 25355 and 12350 high density polyethylene (“HDPE”). Moreover, other suitable polyolefins may include polypropylenes, such as Exxon Chemical Company's Escorene® PD 3445 polypropylene and Montell Chemical Co.'s PF-304 and PF-015.

Further, some suitable polyamides may be found in “Polymer Resins” by Don E. Floyd (Library of Congress Catalog No. 66-20811, Reinhold Publishing, N.Y., 1966). Commercially available polyamides that can be used include Nylon-6, Nylon 6,6, Nylon-11 and Nylon-12. These polyamides are available from a number of sources, such as Emser Industries of Sumter, S.C. (Grilon® & Grilamid® nylons), Atochem Inc. Polymers Division of Glen Rock, N.J. (Rilsan® nylons), Nyltech of Manchester, N.H. (grade 2169, Nylon 6), and Custom Resins of Henderson, Ky. (Nylene 401-D), among others.

In some embodiments, bicomponent fibers can also be utilized. Bicomponent fibers are fibers that can contain two materials such as but not limited to in a side by side arrangement, in a matrix-fibril arrangement wherein a core polymer has a complex cross-sectional shape, or in a core and sheath arrangement. In a core and sheath fiber, generally the sheath polymer has a lower melting temperature than the core polymer to facilitate thermal bonding of the fibers. For instance, the core polymer, in one embodiment, can be nylon or a polyester, while the sheath polymer can be a polyolefin such as polyethylene or polypropylene. Such commercially available bicomponent fibers include “CELBOND” fibers marketed by the Hoechst Celanese Company.

As stated above, one or more films may also be utilized in forming a substrate of the laminate structure of the present invention. In some instances, the thickness of the films may be selected within a certain range to enhance the flexibility of the laminate structure. For example, as stated above, an increase in the thickness of a particular substrate can cause the stiffness of the substrate to increase to the third power with thickness. Thus, in some embodiments, the thickness of the films can be less than about 0.05 inches, in some embodiments between about 0.0003 inches to about 0.01 inches, and in some embodiments, between about 0.0007 inches to about 0.02 inches.

To form the films, a variety of materials can be utilized. For instance, some suitable thermoplastic polymers used in the fabrication of films can include, but are not limited to, polyolefins (e.g., polyethylene, polypropylene, etc.), including homopolymers, copolymers, terpolymers and blends thereof; ethylene vinyl acetate; ethylene ethyl acrylate; ethylene acrylic acid; ethylene methyl acrylate; ethylene normal butyl acrylate; polyurethane; poly(ether-ester); poly(amid-ether) block copolymers; and the like.

The permeability of a substrate utilized in the present invention can also be varied for a particular application. For example, in some embodiments, one or more of the substrates can be permeable to liquids. Such substrates, for example, may be useful in various types of fluid absorption and filtration applications. In other embodiments, one or more of the substrates can be impermeable to liquids, such as films formed from polypropylene or polyethylene. In addition, in other embodiments, it may be desired that one or more of the substrates be impermeable to liquids, but permeable to gases and water vapor (i.e., breathable).

For instance, some suitable breathable, liquid-impermeable substrates can include substrates such as disclosed in U.S. Pat. No. 4,828,556 to Braun et al., which is incorporated herein in its entirety by reference thereto for all purposes. The breathable substrate of Braun et al. is a multilayered, cloth-like barrier that includes at least three layers. The first layer is a porous nonwoven web; the second layer, which is joined to one side of the first layer, contains a continuous film of polyvinyl alcohol; and the third layer, which is joined to either the second layer or the other side of the first layer not joined with the second layer, contains another porous nonwoven web. The second layer of continuous film of polyvinyl alcohol is not microporous, meaning that it is substantially free of voids which connect the upper and lower surfaces of the film.

In other cases, various substrates can be constructed with films containing micropores to provide breathability to the substrate. The micropores form what is often referred to as “tortuous pathways” through the film. Specifically, liquids contacting one side of the film do not have a direct passage through the film. Instead, a network of microporous channels in the film prevents liquid water from passing, but allows water vapor to pass.

In some instances, the breathable, liquid-impermeable substrates are made from polymer films that contain any suitable substance, such as calcium carbonate. The films are made breathable by stretching the filled films to create the microporous passageways as the polymer breaks away from the calcium carbonate during stretching.

Another example of a breathable, yet liquid-impermeable substrate is described in U.S. Pat. No. 5,591,510 to Junker et al., which is incorporated herein in its entirety by reference thereto for all purposes. The fabric material described in Junker et al. contains a breathable outer layer of paper stock and a layer of breathable, fluid-resistant nonwoven material. The fabric also includes a thermoplastic film having a plurality of perforations which allow the film to be breathable while resisting direct flow of liquid therethrough.

In addition to the substrates mentioned above, various other breathable substrates can be utilized. For instance, one type of substrate that may be used is a nonporous, continuous film, which, because of its molecular structure, is capable of forming a vapor-permeable barrier. For example, among the various polymeric films that may fall into this type include films made from a sufficient amount of poly(vinyl alcohol), polyvinyl acetate, ethylene vinyl alcohol, polyurethane, ethylene methyl acrylate, and ethylene methyl acrylic acid to make them breathable.

Still, other breathable substrates that can be used in the present invention include apertured films. For instance, in one embodiment, an apertured film can be used that is made from a thermoplastic film, such as polyethylene, polypropylene, copolymers of polypropylene or polyethylene, or calcium carbonate-filled films. The particular aperturing techniques utilized to obtain the apertured film layer may be varied. The film may be formed as an apertured film or may be formed as a continuous, non-apertured film and then subjected to a mechanical aperturing process.

Moreover, in some embodiments, one or more of the substrates used in the laminate structure can contain an elastomeric component that includes at least one elastomeric material. For example, an elastomeric or elastic material can refer to material that, upon application of a force, is stretchable to a stretched, biased length which is at least about 150%, or one and a half times, its relaxed, unstretched length, and which will recover at least about 50% of its elongation upon release of the stretching, biasing force. In some instances, an elastomeric component can enhance the flexibility of the resulting laminate structure by enabling the structure to be more easily bent and distorted. Moreover, in other embodiments, the elastomeric component can also allow the particles to swell to a greater extent by allowing the substrates to more easily distort. In particular, the use of an elastomeric material may, in some embodiments, increase the amount of force required to rupture the substrate.

When present in a substrate, the elastomeric component can take on various forms. For example, the elastomeric component can make up the entire substrate or form a portion of the substrate. In some embodiments, for instance, the elastomeric component can contain elastic strands or sections uniformly or randomly distributed throughout the substrate. Alternatively, the elastomeric component can be an elastic film or an elastic nonwoven web. The elastomeric component can also be a single layer or a multi-layered material.

In general, any material known in the art to possess elastomeric characteristics can be used in the present invention in the elastomeric component. For example, suitable elastomeric resins include block copolymers having the general formula A-B-A′ or A-B, where A and A′ are each a thermoplastic polymer endblock which contains a styrenic moiety such as a poly(vinyl arene) and where B is an elastomeric polymer midblock such as a conjugated diene or a lower alkene polymer. Block copolymers for the A and A′ blocks, and the present block copolymers are intended to embrace linear, branched and radial block copolymers. In this regard, the radial block copolymers may be designated (A-B)m-X, wherein X is a polyfunctional atom or molecule and in which each (A-B)m- radiates from X in a way that A is an endblock. In the radial block copolymer, X may be an organic or inorganic polyfunctional atom or molecule and m may be an integer having the same value as the functional group originally present in X, which is usually at least 3, and is frequently 4 or 5, but not limited thereto. Thus, the expression “block copolymer,” and particularly “A-B-A” and “A-B” block copolymers, can include all block copolymers having such rubbery blocks and thermoplastic blocks as discussed above, which can be extruded (e.g., by meltblowing), and without limitation as to the number of blocks. For example, elastomeric materials, such as (polystyrene/poly(ethylene-butylene)/polystyrene) block copolymers, can be utilized. Commercial examples of such elastomeric copolymers are, for example, those known as KRATON® materials which are available from Shell Chemical Company of Houston, Tex. KRATON® block copolymers are available in several different formulations, a number of which are identified in U.S. Pat. Nos. 4,663,220, 4,323,534, 4,834,738, 5,093,422 and 5,304,599, which are hereby incorporated in their entirety by reference thereto for all purposes.

Polymers composed of an elastomeric A-B-A-B tetrablock copolymer may also be used. Such polymers are discussed in U.S. Pat. No. 5,332,613 to Taylor et al. In these polymers, A is a thermoplastic polymer block and B is an isoprene monomer unit hydrogenated to substantially a poly(ethylene-propylene) monomer unit. An example of such a tetrablock copolymer is a styrene-poly(ethylene-propylene)-styrene-poly(ethylene-propylene) or S-EP-S-EP elastomeric block copolymer available from the Shell Chemical Company of Houston, Tex. under the trade designation KRATON® G-1657.

Other exemplary elastomeric materials that may be used include polyurethane elastomeric materials such as, for example, those available under the trademark ESTANE® from B. F. Goodrich & Co. or MORTHANE® from Morton Thiokol Corp., and polyester elastomeric materials such as, for example, copolyesters available under the trade designation HYTREL® from E. I. DuPont De Nemours & Company and copolyesters known as ARNITEL®, formerly available from Akzo Plastics of Amhem, Holland and now available from DSM of Sittard, Holland.

Another suitable material is a polyester block amide copolymer having the formula:

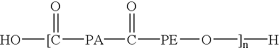

where n is a positive integer, PA represents a polyamide polymer segment and PE represents a polyether polymer segment. In particular, the polyether block amide copolymer has a melting point of from about 150° C. to about 170°C., as measured in accordance with ASTM D-789; a melt index of from about 6 grams per 10 minutes to about 25 grams per 10 minutes, as measured in accordance with ASTM D-1238, condition Q (235 C/1 Kg load); a modulus of elasticity in flexure of from about 20 Mpa to about 200 Mpa, as measured in accordance with ASTM D-790; a tensile strength at break of from about 29 Mpa to about 33 Mpa as measured in accordance with ASTM D-638 and an ultimate elongation at break of from about 500 percent to about 700 percent as measured by ASTM D-638. A particular embodiment of the polyether block amide copolymer has a melting point of about 152° C. as measured in accordance with ASTM D-789; a melt index of about 7 grams per 10 minutes, as measured in accordance with ASTM D-1238, condition Q (235 C/1 Kg load); a modulus of elasticity in flexure of about 29.50 Mpa, as measured in accordance with ASTM D-790; a tensile strength at break of about 29 Mpa, as measured in accordance with ASTM D-639; and an elongation at break of about 650 percent, as measured in accordance with ASTM D-638. Such materials are available in various grades under the trade designation PEBAX® from ELF Atochem Inc. of Glen Rock, N.J. Examples of the use of such polymers may be found in U.S. Pat. Nos. 4,724,184, 4,820,572 and 4,923,742 to Killian.

Elastomeric polymers can also include copolymers of ethylene and at least one vinyl monomer such as, for example, vinyl acetates, unsaturated aliphatic monocarboxylic acids, and esters of such monocarboxylic acids. The elastomeric copolymers and formation of elastomeric nonwoven webs from those elastomeric copolymers are disclosed in, for example, U.S. Pat. No.4,803,117.

The thermoplastic copolyester elastomers include copolyetheresters having the general formula:

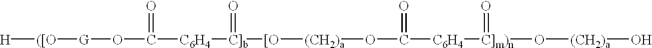

where “G” is selected from the group consisting of poly(oxyethylene)-alpha, omega-diol, poly(oxypropylene)-alpha, omega-diol, poly(oxytetramethylene)-alpha, omega-diol and “a” and “b” are positive integers including 2, 4 and 6, “m” and “n” are positive integers including 1-20. Such materials generally have an elongation at break of from about 600 percent to 750 percent when measured in accordance with ASTM D-638 and a melt point of from about 350° F. to about 400° F. (176 to 205° C.) when measured in accordance with ASTM D-2117.

In addition, some examples of suitable elastomeric olefin polymers are available from Exxon Chemical Company of Baytown, Tex. under the trade name ACHIEVE® for polypropylene based polymers and EXACT® and EXCEED® for polyethylene based polymers. Dow Chemical Company of Midland, Mich. has polymers commercially available under the name ENGAGE®. These materials are believed to be produced using non-stereoselective metallocene catalysts. Exxon generally refers to their metallocene catalyst technology as “single site” catalysts, while Dow refers to theirs as “constrained geometry” catalysts under the name INSIGHT® to distinguish them from traditional Ziegler-Natta catalysts which have multiple reaction sites.

When incorporating an elastomeric component containing an elastomeric material, such as described above, into a substrate, it is sometimes desired that the elastomeric component be an elastic laminate that contains an elastomeric material with one or more other layers, such as foams, films, apertured films, and/or nonwoven webs. An elastic laminate generally contains layers that can be bonded together so that at least one of the layers has the characteristics of an elastic polymer. The elastic material used in the elastic laminates can be made from materials, such as described above, that are formed into films, such as a microporous film, fibrous webs, such as a web made from meltblown fibers, spunbond fibers, foams, and the like.

For example, in one embodiment, the elastic laminate can be a “neck-bonded” laminate. A “neck-bonded” laminate refers to a composite material having at least two layers in which one layer is a necked, nonelastic layer and the other layer is an elastic layer. The resulting laminate is thereby a material that is elastic in the cross-direction. Some examples of neck-bonded laminates are described in U.S. Pat. Nos. 5,226,992, 4,981,747, 4,965,122, and 5,336,545, all to Morman, all of which are incorporated herein in their entirety by reference thereto for all purposes.

The elastic laminate can also be a “stretch-bonded” laminate, which refers to a composite material having at least two layers in which one layer is a gatherable layer and in which the other layer is an elastic layer. The layers are joined together when the elastic layer is in an extended condition so that upon relaxing the layers, the gatherable layer is gathered. For example, one elastic member can be bonded to another member while the elastic member is extended at least about 25 percent of its relaxed length. Such a multilayer composite elastic material may be stretched until the nonelastic layer is fully extended.

For example, one suitable type of stretch-bonded laminate is a spunbonded laminate, such as disclosed in U.S. Pat. No. 4,720,415 to VanderWielen et al., which is incorporated herein in its entirety by reference thereto for all purposes. Another suitable type of stretch-bonded laminate is a continuous filament spunbonded laminate, such as disclosed in U.S. Pat. No. 5,385,775 to Wright, which is incorporated herein in its entirety by reference thereto for all purposes. For instance, Wright discloses a composite elastic material that includes: (1) an anisotropic elastic fibrous web having at least one layer of elastomeric meltblown fibers and at least one layer of elastomeric filaments autogenously bonded to at least a portion of the elastomeric meltblown fibers, and (2) at least one gatherable layer joined at spaced-apart locations to the anisotropic elastic fibrous web so that the gatherable layer is gathered between the spaced-apart locations. The gatherable layer is joined to the elastic fibrous web when the elastic web is in a stretched condition so that when the elastic web relaxes, the gatherable layer gathers between the spaced-apart bonding locations. Other composite elastic materials are described and disclosed in U.S. Pat. Nos. 4,789,699 to Kieffer et al., 4,781,966 to Taylor, 4,657,802 to Morman, and 4,655,760 to Morman et al., all of which are incorporated herein in their entirety by reference thereto for all purposes.

In one embodiment, the elastic laminate can also be a necked stretch bonded laminate. As used herein, a necked stretch bonded laminate is defined as a laminate made from the combination of a neck-bonded laminate and a stretch-bonded laminate. Examples of necked stretch bonded laminates are disclosed in U.S. Pat. Nos. 5,114,781 and 5,116,662, which are both incorporated herein in their entirety by reference thereto for all purposes. Of particular advantage, a necked stretch bonded laminate can be stretchable in both the machine and cross-machine directions.

In some embodiments, the material(s) used in forming a substrate of the present invention can provide a “light scattering” effect to mask the color of particles contained therein. For example, as described in more detail below, particles having a certain color may be utilized. In many applications, it may be desired that the color not be seen through the resulting laminate structure. Thus, in accordance with one embodiment of the present invention, the substrates can be formed and bonded to other substrates in a manner so that the color of the particles is substantially masked. For example, in one embodiment, meltblown nonwoven webs formed from synthetic fibers can be utilized as the substrates with black particles (e.g., activated carbon) sandwiched therebetween. In this embodiment, the fine fibrous network of the meltblown nonwoven substrates can substantially mask the color of the particles contained within the pockets of the laminate structure.

In accordance with the present invention, as stated above, particles are also provided for deposition onto one or more of the substrates. The particles may be chemically reactive or inert. In general, the particles may be of any size, shape, and/or type. For example, the particles may be spherical or semispherical, cubic, rod-like, polyhedral, etc., while also including other shapes, such as needles, flakes, and fibers. Moreover, some examples of suitable particles can include, but are not limited to, superabsorbents, deodorants, colorants (e.g., encapsulated dyes), fragrances, catalysts, germicidal materials, filtration media (e.g., activated carbon), proteins, drug particles, etc. For example, the particles may be selected from inorganic solids, organic solids, etc. Some inorganic solids that can be utilized include, but are not limited to, silicas, metals, metal complexes, metal oxides, zeolites and clays. Moreover, some examples of suitable organic solids that can be utilized include, but are not limited to, activated carbons, activated charcoals, molecular sieves, polymer microsponges, polyacrylates, polyesters, polyolefins, polyvinyl alcohols, and polyvinylidine halides. Other solids that can be used may include pulp materials, such as microcrystalline cellulose, highly refined cellulose pulp, bacterial cellulose, and the like.

The particles can be deposited onto the substrate using a variety of deposition techniques. For instance, in some embodiments, a template can be utilized to deposit the particles in a desired pattern. Specifically, a template can have a structure that enables it to physically inhibit the areas that are to be bonded from being deposited with the particles. In addition, in some embodiments, vacuum plates can be utilized. Vacuum plates use suctional forces to draw the particles to the desired areas. Moreover, adhesive particle deposition can also be used. For example, an adhesive can be applied to the substrate where it is desired for the particles to be deposited. The particles will then adhere to those portions of the substrate containing the adhesive.

Further, in some embodiments, one or more of the substrates can be textured such that the substrate contains depressions and elevations. In such instances, particles can be deposited onto the textured substrate such that they collect substantially in the depressions of the substrate. Besides the above-mentioned techniques of particle deposition, other techniques can also be utilized. For instance, some other known techniques for depositing particles onto a substrate can include, but are not limited to, electrostatic, xerographic, printing (e.g., gravure), patterned transfer roll (vacuum or adhesive), and the like.

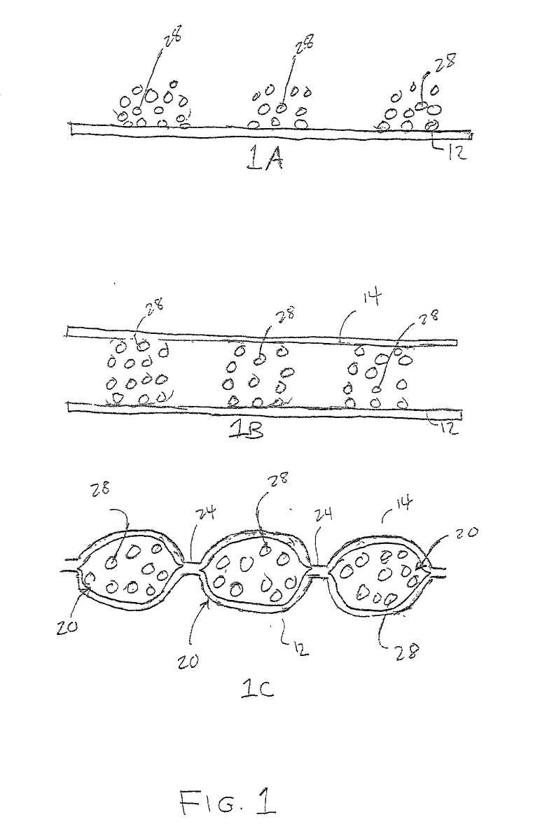

For instance, referring to FIG. 1, one embodiment of a method for enclosing particles within a laminate structure 10 is illustrated. As shown in FIG. 1A, the particles are initially deposited onto a first substrate 12. Once deposited, a second substrate 14 can then be bonded to portions of the first substrate 12.

In accordance with the present invention, the substrates are generally bonded together only at those regions on which the particles have not been deposited. For example, as shown in FIGS. 1B-1C, in one embodiment, the substrate 14 can be bonded to the first substrate 12 at certain bonded portions 24. As a result, discrete regions of particles 28 can be contained within unbonded portions or pockets 20. In some embodiments, these pockets 20 can provide substantial benefits to the resulting laminate structure. For instance, when utilizing a laminate structure that is designed to be an absorbent article, such as a diaper, it may be desired to direct the flow of liquids to discrete regions of superabsorbent particles for absorbing the liquids. Thus, in such instances, the bonded portions of the laminate structure can be formed from certain materials, such as films or nonwoven webs, that are or become substantially impermeable to liquids when bonded together. However, the unbonded portions of the substrates can remain substantially permeable to liquids such that any liquid contacting the laminate structure is primarily directed to the unfused portions or pockets of the laminate structure so that they contact the discrete regions of superabsorbent particles. However, it should also be understood that the laminate structure of the present invention is not limited to any particular application. In fact, virtually any type of particle can be incorporated into the pockets of the laminate structure so that the resulting laminate can be used in a wide variety of applications.

The pockets 20 can generally have a variety of different sizes and/or shapes. For example, the pockets 20 can have regular or irregular shapes. Some regular shapes can include, for example, circles, ovals, squares, ellipses, hexagons, rectangles, hourglass-shaped, tube-shaped, etc. Moreover, in some instances, some pockets of the laminate structure may have different shapes and/or sizes than other pockets.

To bond the substrates together in a manner such as described above, a variety of methods can be utilized. In particular, any method that allows the substrates to be bonded together in a pattern corresponding to the portions of the substrate that do not contain the discrete regions of the particles can be utilized. For instance, thermal bonding techniques, such as thermal point bonding, pattern unbending, etc., and ultrasonic bonding are some examples of techniques that may be utilized in the present invention to fuse together the substrates. In addition, other bonding methods, such as adhesive bonding, etc., may also be utilized to bond together the substrates. For example, some suitable adhesives are described in U.S. Pat. Nos. 5,425,725 to Tanzer, et al.; 5,433,715 to Tanzer, et al.; and 5,593,399 to Tanzer, et al., which are incorporated herein in their entirety by reference thereto for all purposes.

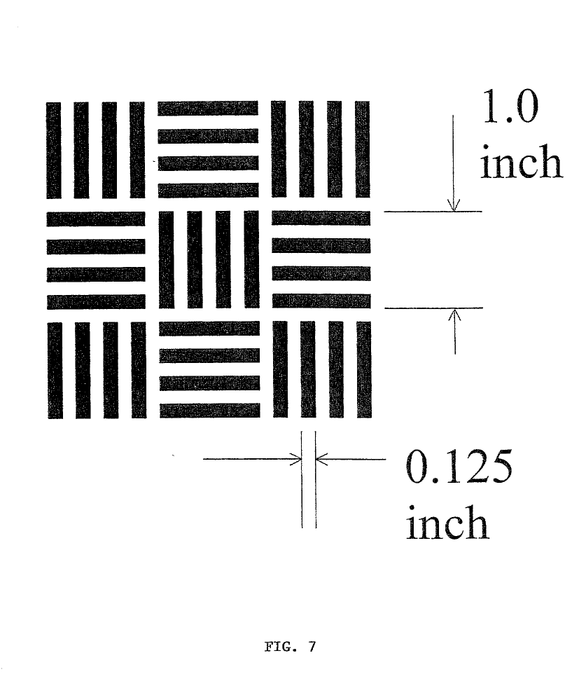

Referring to FIG. 6, one particular embodiment for bonding the second substrate 14 to the substrate 12 is illustrated. As shown, the particles 28 are first deposited by a dispenser 35 onto the substrate 12 in a preselected pattern. The substrate 12 is moved under the dispenser 35 with the aid of a roll 37. Further, in this embodiment, to facilitate deposition of the particles 28 onto the substrate 12, a vacuum roll 33 is utilized. In particular, the vacuum roll 33 can apply a suctional force to the lower surface of the substrate 12 to better control the positioning of the particles 28 within a discrete region of the substrate 12.

Thereafter, the substrate 12 containing the particles 28 is passed beneath the substrate 14. In this embodiment, each substrate 12 and 14 contains a heat-fusible material, such as polypropylene. As shown, the substrates 12 and 14 are passed under a roll 30 that is heated and contains a surface having various protrusions 32. The protrusions 32 form a pattern that corresponds to portions of the substrate 12 that do not contain the particles 28. In this embodiment, another heated roll 34 that has a smooth surface is also utilized to facilitate the fusing of the substrates 12 and 14. However, it should be understood that the roll 34 is not required in all instances. Moreover, the roll 34 may also have a certain pattern of protrusions and/or may remain unheated. In the illustrated embodiment, as the heated rolls 30 and 34 press the fusible substrates 12 and 14, the areas at the protrusions 32 are fused together, forming fused portions surrounding the pockets (i.e., unfused portions) containing the particles.

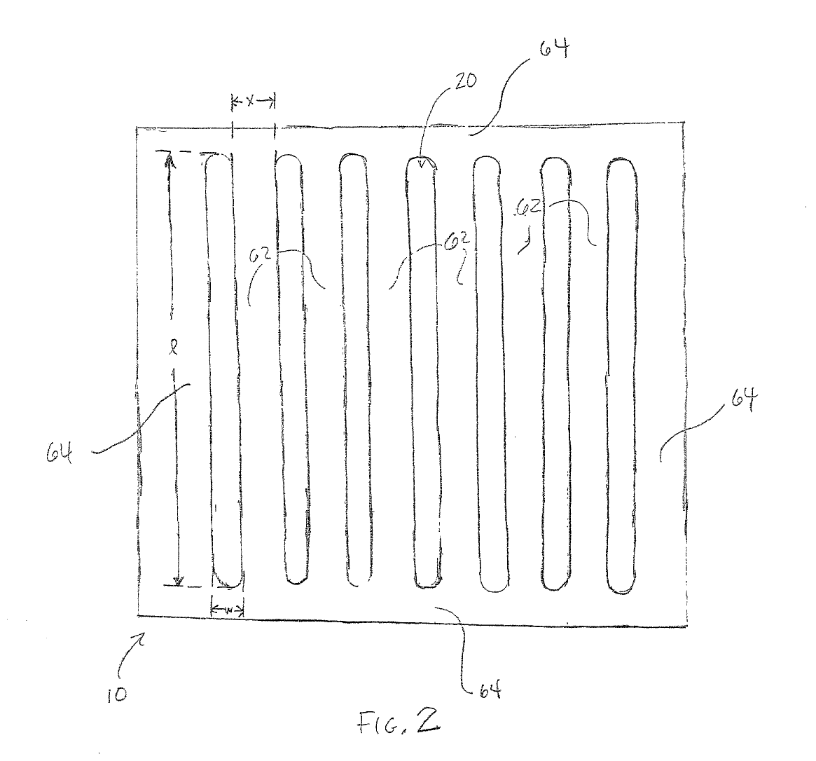

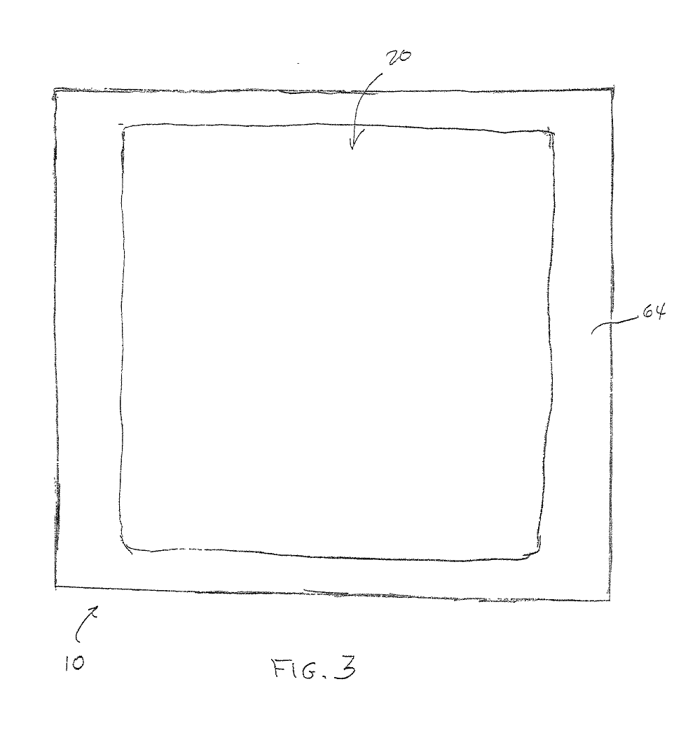

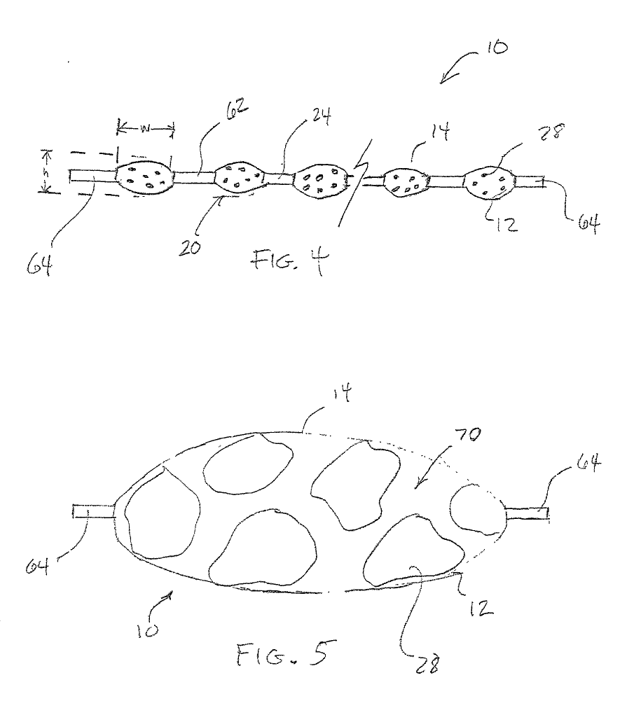

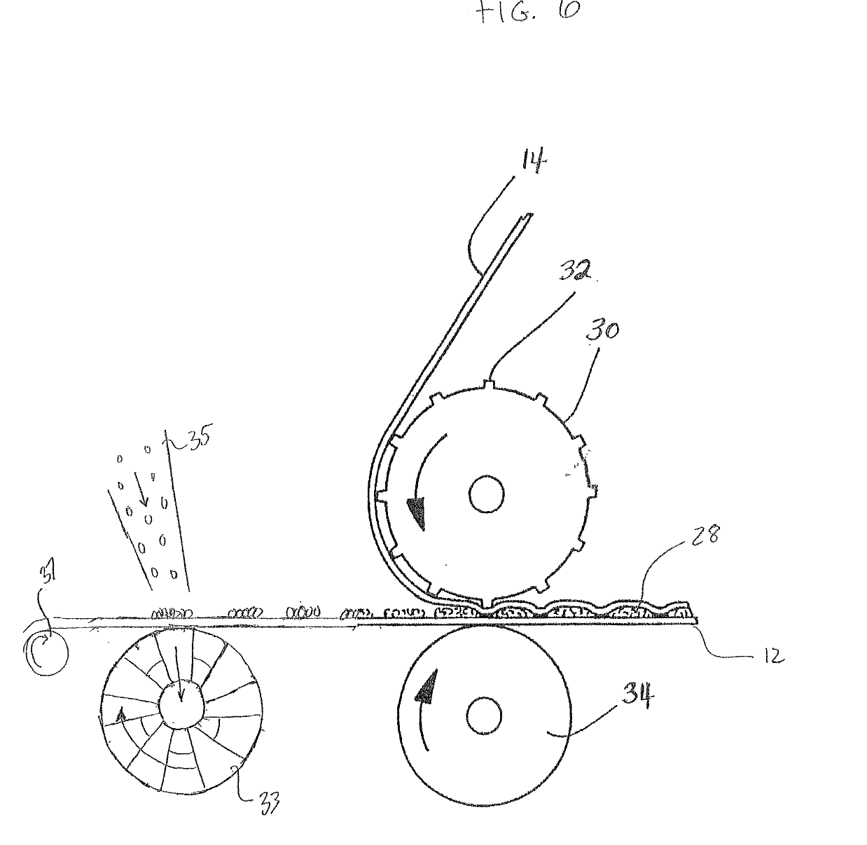

The bonding strength(s) attained by bonding the substrates 12 and 14 together, such as described above, can generally be varied based on the particular application and amount of force that the swelling of a particle may apply. For instance, referring to FIGS. 2 and 4, one embodiment of a laminate structure 10 is illustrated that contains bonded portions 24 (FIG. 1) defining perimeter regions 64 and inner regions 62. The inner regions 62 are typically bonded to an extent such that, upon sufficient swelling of the particles 28 when contacted with a liquid, the inner regions 62 can delaminate at a controlled rate. For example, in one embodiment, the inner regions 62 are thermally bonded using a Carver press having platens with a surface area of 144 inches². In particular, the press applies a pressure of 10,000 pounds per square inch to a 36-inch square pattern plate (40% open area) for 60 seconds at a temperature of 140° C. Thus, as shown in FIGS. 3 and 5, for example, the delamination of the inner regions 62 creates a single, large pocket 70 containing the particles 28. The pocket 70 provides an increased volume through which the particles 28 can expand. As such, particles 28 that were not previously utilized (e.g., unswollen) because of geometrical obstacles can be readily exposed to the liquid.

The extent of bonding of the inner regions 62 and/or the perimeter regions 64 can generally be varied as desired. For example, in some embodiments, the extent of bonding for the perimeter regions 64 can approximate the extent of bonding of for the inner regions 62. Moreover, the bond width of the perimeter regions 64 may, if desired, be greater than the bond width for the inner regions 62. For example, the bond width for the perimeter regions 64 can, in one embodiment, be about 0.60 inches, while the bond width for the inner regions 62 can be about 0.10 inches. By having a greater bond width, the perimeter regions 64 can partially delaminate upon the application of a force thereto without substantially delaminating. In particular, inner regions 62 having a bond width of about 0.10 inches may completely delaminate upon the application of a certain force, while perimeter regions 64 having a bond width of about 0.60 inches may only delaminate about 0.10 inches (the bond width of the inner regions 62).

In addition, in some embodiments, it may also be desired to vary the extent of bonding throughout the bonded regions of the laminate structure. For example, in some embodiments, the perimeter regions 64 can be bonded to a greater extent (e.g., higher temperatures, higher pressures, longer bonding times, etc.) than the inner regions 62. Such increased bonding for the perimeter regions 64 can further ensure that the perimeter regions 64 do not substantially delaminate upon the application of a force. For example, in one embodiment, the perimeter regions 64 are bonded to an extent such that the bonding strength of the perimeter regions 64 approximates the strength of the substrate 12 and/or the substrate 14. As a result, the substrates 12 and 14 will not typically completely delaminate upon the swelling of particles 28.

In some instances, it may also be desired to control the proportions of the bonded surface area and the unbonded surface area. For example, in some embodiments, the bonded surface area can be between about 10% to about 500% of the unbonded surface area, in some embodiments, between about 10% to about 100% of the unbonded surface area, and in some embodiments, between about 40% to about 60% of the unbonded surface area.

To facilitate delamination of the inner regions 62 upon the application of a certain force, the pockets 20 can also be formed to have a certain size and/or shape. For example, in some embodiments, the pockets 20 can be elongated. In particular, elongated pockets typically have a length “l” to width “w” ratio (i.e., l/w) of greater than about 2, in some embodiments between about 4 to about 100, and in some embodiments, between about 6 to about 10. For example, the length dimension “l” of the pockets 20 can, in some embodiments, be less than about 2 inches, in some embodiments between about 0.0625 inches to about 2 inches, and in some embodiments, between about 0.25 inches to about 2 inches.

By providing elongated pockets having a certain length-to-width ratio, such as set forth above, it has been discovered that the inner regions may delaminate more readily at a controlled rate upon the application of a force. In particular, it is believed that elongated pockets allow for forces created by the swelling of a particle to be applied to a greater extent in the width “w” direction of the pockets 20 than in the length “l” direction of the pockets 20, thereby further facilitating the ability of the pockets 20 to delaminate in the manner illustrated in FIG. 5. For instance, as shown in FIGS. 2-5, as the particles 28 contained within the pockets 20 begin to swell, they exert pressure on the perimeter regions 64 and the inner regions 62 of the laminate structure 10. However, because the pockets 20 are elongated, a greater force is believed to be exerted in the width “w” direction of the inner regions 62, thereby causing such regions to rupture before the perimeter regions 64. Thus, although, as described above, the perimeter regions 64 are typically bonded to a greater extent than the inner regions 62, the perimeter regions 64 may be bonded to a lesser extent than might be required for pockets having other shapes and/or sizes.

In addition, the spacing between the pockets can also be varied. For example, in some instances, pockets that are spaced relatively close together may delaminate more readily than pockets spaced relatively far apart. Thus, as shown in FIG. 2, the approximate maximum distance “x” that the pockets 20 are spaced apart can, in some embodiments, be greater than about 0.0625 inches, in some embodiments between about 0.0625 inches to about 0.5 inches, and in some embodiments, between about 0.125 inches to about 0.25 inches.

Besides having a certain length-to-width ratio and spacing between the pockets, the boundaries for length, width, and height of the pockets can also fall within a certain range such that the resulting pockets are relatively small and allow flexibility of the resulting laminate structure. For example, referring to FIG. 2, the approximate width “w” to height “h” ratio of the pockets 20 (i.e., w/h) before delamination can, in some embodiments, be less than 10, in some embodiments between about 1 to about 8, and in some embodiments, between 1 to about 5. For example, in some embodiments, the approximate height “h” before delamination can be equal to less than about 1 inch, in some embodiments less than about 0.5 inches, and in some embodiments, between about 0.005 inches to about 0.4 inches.

Although various dimensions have been set forth above, it should understood that other dimensions are also contemplated in the present invention. For instance, the particular pocket dimensions may vary depending on the overall dimensions of the laminate structure. Moreover, it should also be understood that the dimensions set forth above are approximate “maximum” or “minimum” dimensions for a given direction. Thus, a pocket having a certain approximate height, for example, may have other heights at different locations in the width direction of the pocket. In some instances, some of the heights of a pocket may actually exceed the given dimension by a relatively small amount.

The present invention may be better understood with reference to the following examples.

### EXAMPLE 1

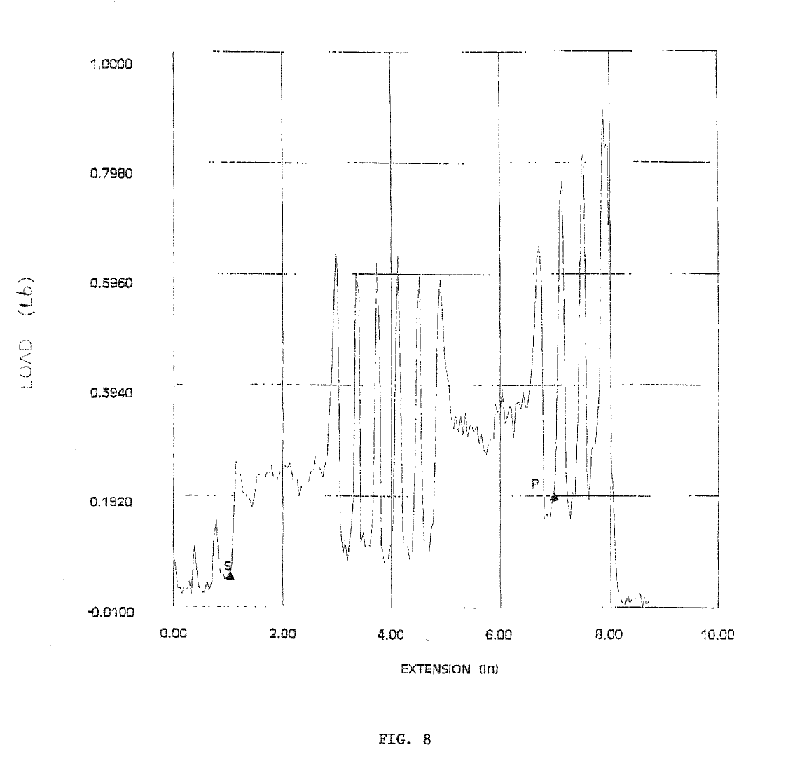

The ability to form a laminate structure that is capable of delamination was demonstrated. Initially, two (2) polypropylene meltblown sheets, each having a basis weight of 2 ounces per square yard, were thermally laminated together for 60 seconds at a hydraulic pressure of 15,000 pounds per square inch and a temperature of 150° C. using a Carver 12″×12″ heated press. During bonding, a 6″×8″ patterned bonding plate, such as illustrated in FIG. 7, was impressed against the polypropylene sheets to form bonded regions and unbonded regions. Specifically, the regions defined by the unshaded rectangles remained unbonded, while the areas between and around the shaded rectangles, were bonded. The unbonded regions were filled with superabsorbent granules in a manner such as described above, and the bonds around these regions confined the granules.

After being formed, the laminate structure was then contacted with a liquid so that the superabsorbent granules expanded to fill the volume of the unbonded regions. This expansion caused the granules to press against the polypropylene sheets in the z-direction such that pockets having approximately a cylindrical shape were formed to have a diameter of about 0.125 inches and a length of about 1 inch.

Upon continued expansion, the size of the granules began to exceed the volume of the cylindrical pockets until the bonds between parallel pockets delaminated, thereby forming larger square pockets. In particular, the bonded regions delaminated, causing the 0.125-inch wide and 0.125-inch tall pockets to combine into larger pockets having a width of about 1 inch and a height of about 0.5 inches, such as shown in FIGS. 2-3. The edges of the structure also remained bonded, so that the resulting 3-dimensional laminate structure contained the original superabsorbent granules, plus a large amount of liquid.

### EXAMPLE 2

The ability to form a laminate structure that is capable of delamination was demonstrated. Initially, a laminate structure was formed as described in Example 1. To physically demonstrate the ability of the laminate structure to delaminate upon swelling, a grab tensile test was conducted for 6 specimens of the laminate structure. Grab tensile is generally a measure of breaking strength and elongation or strain of a fabric when subjected to a stress. This test is known in the art and conforms to the specifications of ASTM D-5035-95. In the present example, the grab tensile test was performed using two clamps, each having two jaws with each jaw having a facing in contact with one layer of the sample. The clamps held the material in a plane separated by 3 inches and move apart at a constant rate of extension. Values for grab tensile strength and grab elongation were obtained using specimen sizes of 1 inch×4 inches with a jaw facing size of 1 inch by 1 inch, and a constant rate of extension. The specimen was clamped in an Instron Model ™, available from the Instron Corporation, 2500 Washington St., Canton, Mass. 02021.

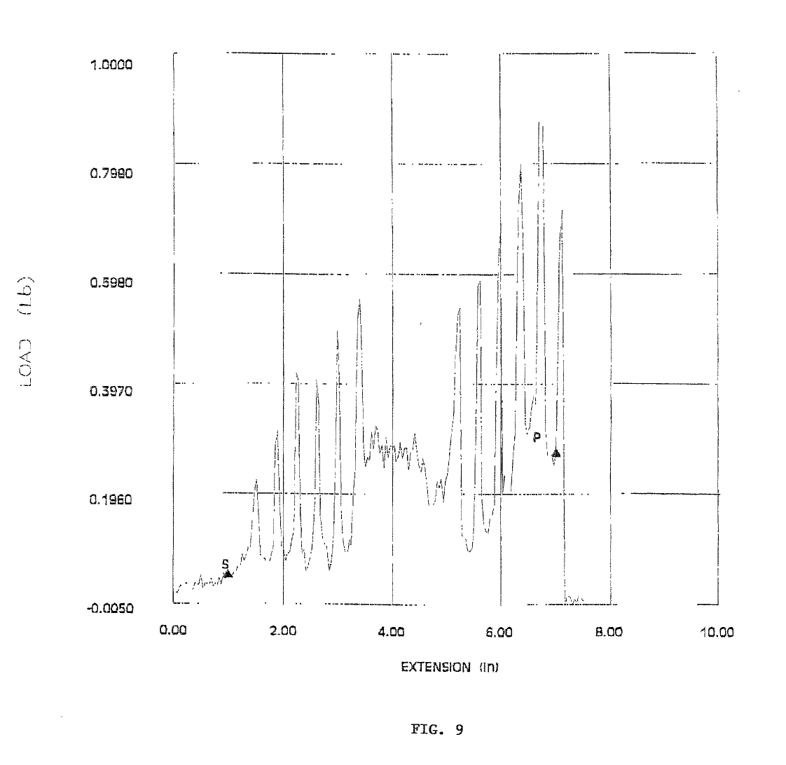

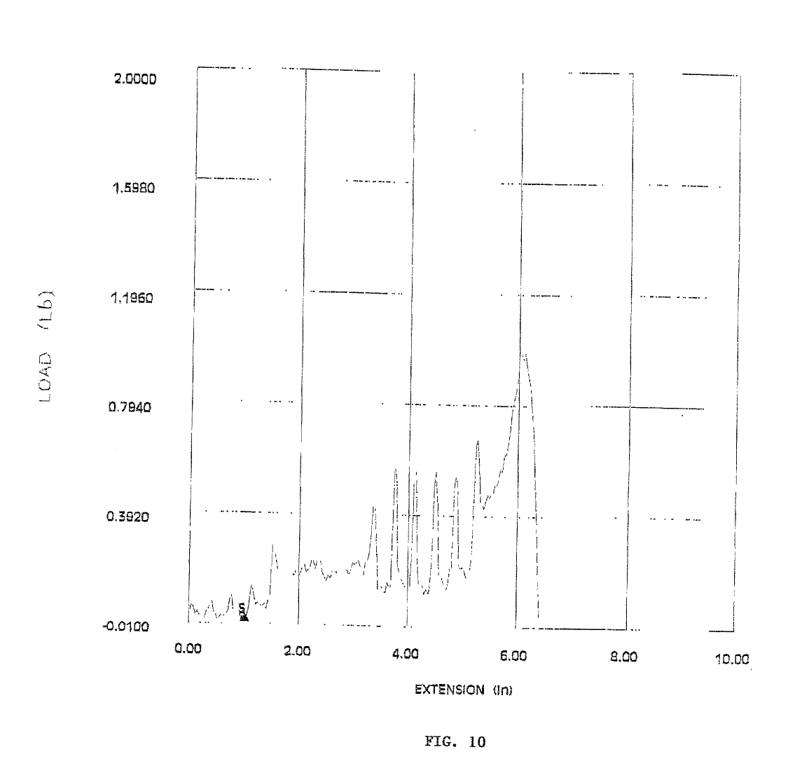

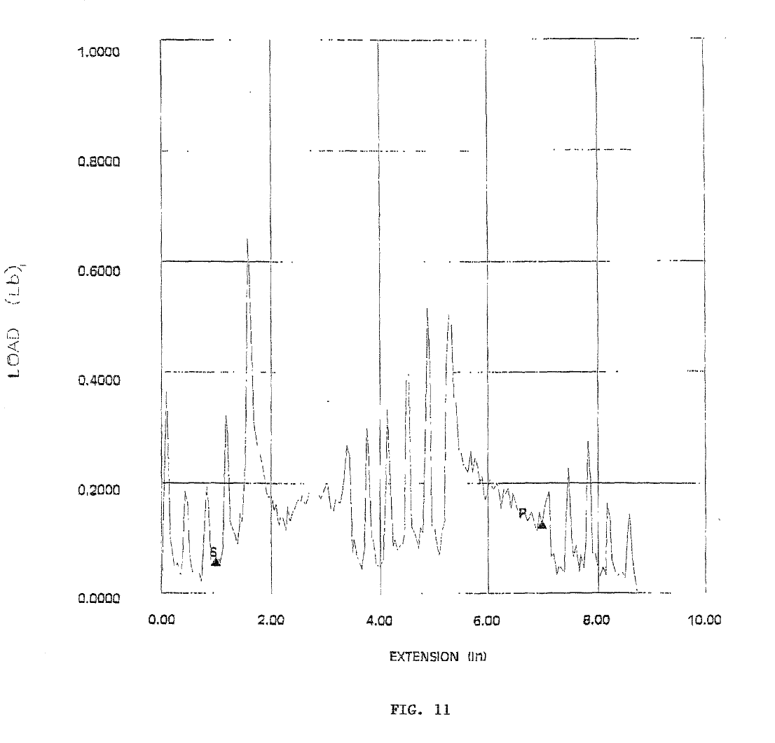

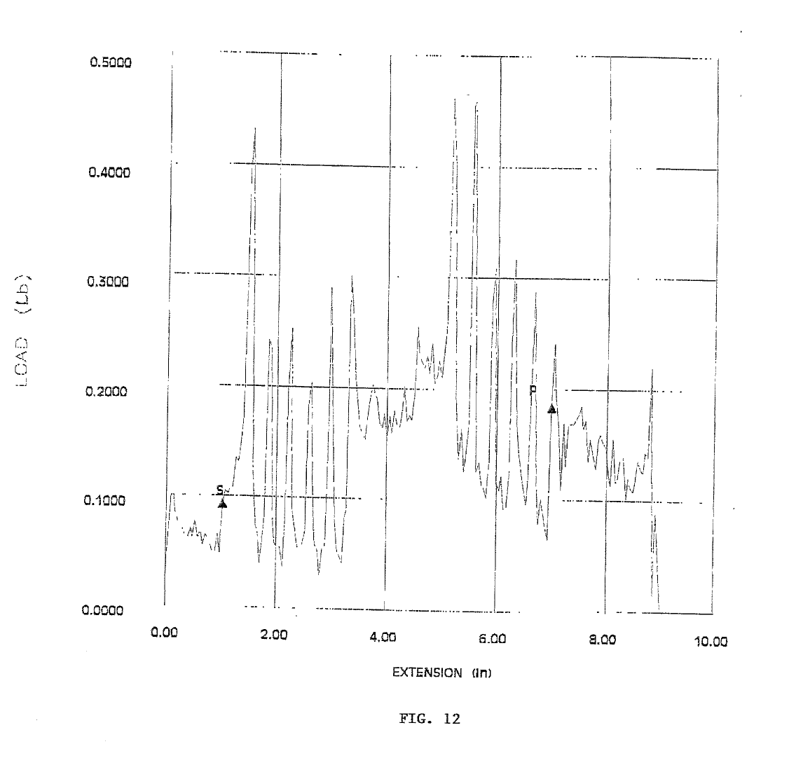

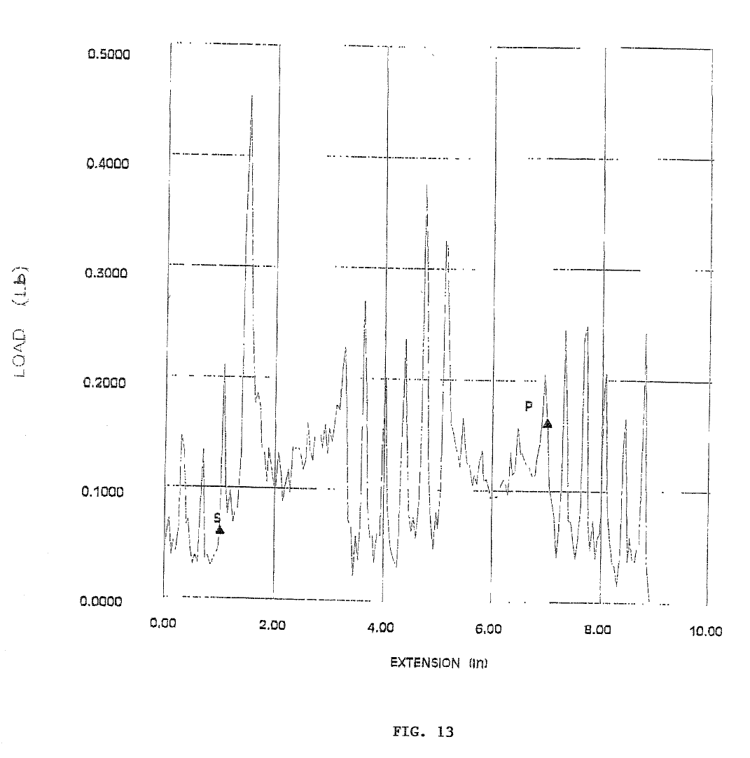

During testing, a stress-strain curve was developed for each specimen to demonstrate the delamination of the laminate structure. The results are expressed as the load (pounds) versus the amount of extension (inches) and are provided in FIGS. 8-13. Referring to FIG. 8, for example, after being extended approximately 1 inch, the specimen delaminated in the lengthwise direction “l” of the pockets, as indicated by the relatively constant load values occurring between about 1 inch to about 2 inches of extension. Moreover, after being extended approximately 2 inches, the specimen delaminated in the width direction “w” of the pockets, as indicated by the alternating peaks and valleys provided in FIGS. 8-13. Upon continued extension past 3 inches, the specimen again delaminated in the lengthwise direction “l” for the next set of pockets, followed by further delamination in the width direction “w”.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A laminate structure comprising:
a first substrate containing a thermoplastic polymer and a second substrate containing a thermoplastic polymer, wherein the thermoplastic polymer of said first substrate is fused together with the thermoplastic polymer of said second substrate to form fused portions and unfused portions located between said fused portions, said unfused portions defining elongated pockets that contain discrete regions of particles, said pockets having a length-to-width ratio of greater than about 2, wherein said fused portions define at least one perimeter region and at least one inner region, said inner region being bonded to an extent such that said inner region is capable of delaminating upon the application of a force thereto, said perimeter region withstanding substantial delamination upon the application of said force.

2. A laminate structure as defined in claim 1, wherein said perimeter region is bonded to a greater extent than said inner region.

3. A laminate structure as defined in claim 1, wherein said perimeter region is bonded to approximately the same extent as said inner region.

4. A laminate structure as defined in claim 1, wherein said perimeter region has a greater bond width than said inner region.

5. A laminate structure as defined in claim 1, wherein the strength of said substrates is such that said force does not cause said substrates to substantially rupture.

6. A laminate structure as defined in claim 5, wherein said perimeter region is bonded to such an extent that the strength of said perimeter region approximates the strength of said substrates.

7. A laminate structure as defined in claim 1, wherein said force is supplied by the swelling of said particles upon being contacted with a liquid.

8. A laminate structure as defined in claim 1, wherein said particles contain a superabsorbent material.

9. A laminate structure as defined in claim 1, wherein said pockets have a length-to-width ratio of between about 4 to about 100.

10. A laminate structure as defined in claim 1, wherein said pockets have a length-to-width ratio of between about 6 to about 10.

11. A laminate structure as defined in claim 1, wherein said pockets have an approximate width-to-height ratio of less than about 10.

12. A laminate structure as defined in claim 1, wherein said pockets have an approximate width-to-height ratio of between about 1 to about 5.

13. A laminate structure as defined in claim 1, wherein at least one of said substrates contains a nonwoven web.

14. A laminate structure as defined in claim 1, wherein at least one of said substrates contains a film.

15. A laminate structure as defined in claim 1, wherein said unfused portions are substantially permeable to liquids and said fused portions are substantially impermeable to liquids.

16. An absorbent article comprising:
a first substrate containing a thermoplastic polymer and a second substrate containing a thermoplastic polymer, wherein the thermoplastic polymer of said first substrate is fused together with the thermoplastic polymer of said second substrate to form fused portions and unfused portions located between said fused portions, wherein said unfused portions define elongated pockets containing discrete regions of a superabsorbent material that is capable of swelling upon being contacted with a liquid, said pockets having a length-to-width ratio of between about 4 to about 100, and wherein said fused portions define at least one perimeter region and at least one inner region, said inner region being bonded to an extent such that said inner region is capable of delaminating upon the application of a force thereto by the swelling of said superabsorbent material, said perimeter region withstanding substantial delamination upon the application of said force.

17. An absorbent article as defined in claim 16, wherein said pockets have a length-to-width ratio between about 6 to about 10.

18. An absorbent article as defined in claim 16, wherein the strength of said substrates is such that said force does not cause said substrates to substantially rupture.

19. An absorbent article as defined in claim 16, wherein said pockets have an approximate width-to-height ratio of less than about 10.

20. An absorbent article as defined in claim 16, wherein said pockets have an approximate width-to-height ratio of between about 1 to about 5.

21. An absorbent article as defined in claim 16, wherein at least one of said substrates contains a material selected from the group consisting of nonwoven webs, films, and combinations thereof.

22. An absorbent article as defined in claim 16, wherein said unfused portions are substantially permeable to liquids and said fused portions are substantially impermeable to liquids.

23. A method for forming a laminate structure comprising:
providing a first substrate containing a thermoplastic polymer;
depositing particles onto said first substrate in discrete regions;
placing a second substrate containing a thermoplastic polymer adjacent said first substrate such that said particles are sandwiched between said first and said second substrates;
fusing the thermoplastic polymer of said first substrate with the thermoplastic polymer of said second substrate to form fused portions and unfused portions located between said fused portions, wherein said unfused portions define elongated pockets containing said discrete regions of particles, said elongated pockets having a length-to-width ratio of greater than about 2, and wherein said fused portions define at least one perimeter region and at least one inner region, said inner region being bonded to an extent such that said inner region is capable of delaminating upon the application of a force thereto, said perimeter region withstanding substantial delamination upon the application of said force.

24. A method as defined in claim 23, wherein said force is supplied by the swelling of said particles upon being contacted with a liquid.

25. A method as defined in claim 23, wherein said particles contain a superabsorbent material.

26. A method as defined in claim 23, wherein said pockets have a length-to-width ratio of between about 4 to about 100.

27. A method as defined in claim 23, wherein said pockets have a length-to-width ratio of between about 6 to about 10.

28. A method as defined in claim 23, wherein the strength of said substrates is such that said force does not cause said substrates to substantially rupture.

29. A method as defined in claim 23, wherein at least one of said substrates contains a material selected from the group consisting of nonwoven webs, films, and combinations thereof.

30. A method as defined in claim 23, wherein said fusing is accomplished by a technique selected from the group consisting of thermal bonding, ultrasonic bonding, adhesive bonding, and combinations thereof.
